(19) United States
(12) Patent Application Publication
(10) Pub. No.: US 2015/0355189 A1
(43) Pub. Date: Dec. 10, 2015

(54) QUANTITATIVE ANALYSIS OF TRANSGENIC PROTEIN 5-ENOLPYRUVYLSHIKIMATE-3-PHOSPHATE SYNTHASE

(71) Applicant: DOW AGROSCIENCES LLC, Indianapolis, IN (US)

(72) Inventors: Trent James Oman, Carmel, IN (US); Barry W. Schafer, Cicero, IN (US); Ryan Christopher Hill, Fishers, IN (US); Jeffrey R. Gilbert, Zionsville, IN (US); Guomin Shan, Carmel, IN (US)

(73) Assignee: DOW AGROSCIENCES LLC, Indianapolis, IN (US)

(21) Appl. No.: 14/719,746

(22) Filed: May 22, 2015

Related U.S. Application Data
(60) Provisional application No. 62/010,113, filed on Jun. 10, 2014.
(60) Provisional application No. 62/010,126, filed on Jun. 10, 2014.
(60) Provisional application No. 62/010,137, filed on Jun. 10, 2014.

(51) Int. Cl. G01N 33/68

(52) U.S. Cl. CPC G01N 33/6848; G01N 2333/91182; G01N 2333/90241; G01N 2333/91051

(57) ABSTRACT

The invention relates to methods for quantitative multiplex analysis of complex protein samples from plants using mass spectroscopy. In some embodiments, the disclosure concerns methods for maintaining a transgenic plant variety, for example by analyzing generations of a transgenic plant variety for selective and sensitive quantitation of multiplexed transgenic proteins.

### CROSS-REFERENCE

This application claims the benefit of priority under 35 U.S.C. §119(e) of U.S. provisional patent application Nos. 62/010,113, 62/010,126, and 62/010,137, all filed Jun. 10, 2014, the contents of which are incorporated by reference in their entireties.

### BACKGROUND OF THE INVENTION

The increasing use of recombinant DNA technology to produce transgenic plants for commercial and industrial use requires the development of high-throughput methods of analyzing transgenic plant lines. Such analytical methods are needed for trait discovery research, product development, seed production, and commercialization and to assist in the rapid development of transgenic plants with desirable or optimal phenotypes. Moreover, current guidelines for the safety assessment of GM plants proposed for human consumption requires characterization at the DNA and protein level between the parent and transformed crop. New plant varieties that are developed consist of increasingly complex genetic modifications including, inter alia, stacked genes, and traits.

The current methods for analysis of transgenic plants that are preferred in the art include DNA-based techniques (for example PCR and/or RT-PCR); the use of reporter genes; Southern blotting; and immunochemistry. All of these methodologies suffer from various shortcomings.

Although mass spectrometry has been disclosed previously, existing approaches are limited without selected and sensitive quantitation. There remains a need for a high-throughput method for selected and sensitive quantitation of products of transgene expression in plants.

### SUMMARY OF THE INVENTION

The invention relates to methods for quantitative multiplex analysis of complex protein samples from plants using mass spectroscopy. In some embodiments, the disclosure concerns methods for maintaining a transgenic plant variety, for example by analyzing generations of a transgenic plant variety for selective and sensitive quantitation of multiplexed transgenic proteins.

In one aspect, provided is a high-throughput method of quantitating one or more protein of interest with known amino acid sequence in a plant-based sample. The method comprises:
(a) extracting proteins from a plant-based sample;
(b) digesting proteins extracted from step (a) to obtain peptides;
(c) separating the peptides in a single step;
(d) determining a plural of signature peptides from the protein of interest with known amino acid sequence;
(e) measuring the plural of signature peptides using high resolution accurate mass spectrometry (HRAM MS); and
(f) quantitating the protein of interest with known amino acid sequence based on measurements of the signature peptides.

In one embodiment, the peptides are separated in a single step by column chromatography. In a further embodiment, the column chromatography comprises a liquid column chromatography. In another embodiment, mass spectral data for the peptides corresponding to the protein of interest are obtained in a single step.

In one embodiment, the one or more protein of interest comprises two proteins of interest. In another embodiment, the one or more protein of interest comprises three to twenty proteins of interest. In another embodiment, the one or more protein of interest comprises three to ten proteins of interest. In another embodiment, the one or more protein of interest comprises four proteins of interest.

In one embodiment, the plant-based sample is from a transgenic plant. In a further embodiment, the one or more protein of interest comprises expected product of transgene expression in the transgenic plant. In another embodiment, the one or more protein of interest comprises a 5′-enolpyruvyl-3′-phosphoshikimate synthase (EPSPS). In another embodiment, the one or more protein of interest comprises 5-enolpyruvylshikimate-3-phosphate synthase (2mEPSPS). In another embodiment, the plural of signature peptides comprises at least one sequence selected from the group consisting of SEQ ID NOs: 2-25. In another embodiment, the plural of signature peptides comprises at least two sequences selected from the group consisting of SEQ ID NOs: 2-25. In another embodiment, the plural of signature peptides comprises at least three sequences selected from the group consisting of SEQ ID NOs: 2-25. In another embodiment, the plural of signature peptides comprises SEQ ID NOs 3, 12, and 21. In another embodiment, the plural of signature peptides consist of SEQ ID NOs 3, 12, and 21.

In another embodiment, the one or more protein of interest comprises an aryloxyalkanoate dioxygenase (AAD). In another embodiment, the one or more protein of interest comprises aryloxyalkanoate dioxygenase-12 (AAD-12). In another embodiment, the plural of signature peptides comprises at least one sequence selected from the group consisting of SEQ ID NOs: 27-45. In another embodiment, the plural of signature peptides comprises at least two sequences selected from the group consisting of SEQ ID NOs: 27-45. In another embodiment, the plural of signature peptides comprises at least three sequences selected from the group consisting of SEQ ID NOs: 27-45. In another embodiment, the plural of signature peptides comprises SEQ ID NOs 28, 29, and 34. In another embodiment, the plural of signature peptides consist of SEQ ID NOs 28, 29, and 34.

In another embodiment, the one or more protein of interest comprises a bialaphos resistance (bar) gene product or phosphinothricin N-acetyltransferase (PAT) enzyme. In another embodiment, the one or more protein of interest comprises phosphinothricin acetyltransferase (PAT). In another embodiment, the plural of signature peptides comprises at least one sequence selected from the group consisting of SEQ ID NOs: 47-60. In another embodiment, the plural of signature peptides comprises at least two sequences selected from the group consisting of SEQ ID NOs: 47-60. In another embodiment, the plural of signature peptides comprises at least three sequences selected from the group consisting of SEQ ID NOs: 47-60. In another embodiment, the plural of signature peptides comprises SEQ ID NOs 49, 55, and 56. In another embodiment, the plural of signature peptides consist of SEQ ID NOs 49, 55, and 56.

In one embodiment, measuring the plural of signature peptides comprises calculating corresponding peak heights or peak areas. In another embodiment, measuring the plural of signature peptides comprises comparing data from high fragmentation mode and low fragmentation mode.

In another aspect, provided is a high-throughput system for quantitating one or more protein of interest with known amino acid sequence in a plant-based sample. The system comprises:
(a) a high-throughput means for extracting proteins from a plant-based sample;
(b) a separation module for separating peptides in a single step;
(c) a selection module for selecting a plural of signature peptides from the protein of interest with known amino acid sequence; and
(d) a high resolution accurate mass spectrometry (HRAM MS) for measuring the plural of signature peptides.

In one embodiment, the separation module comprises a column chromatography. In another embodiment, the column chromatography comprises a liquid column chromatography. In another embodiment, the high resolution accurate mass spectrometry (HRAM MS) comprises a tandem mass spectrometer. In another embodiment, the high resolution accurate mass spectrometry (HRAM MS) does not comprise a tandem mass spectrometer.

In one embodiment, the plant-based sample is from a transgenic plant. In a further embodiment, the one or more protein of interest comprises expected product of transgene expression in the transgenic plant. In another embodiment, the one or more protein of interest comprises a 5′-enolpyruvyl-3′-phosphoshikimate synthase (EPSPS). In another embodiment, the one or more protein of interest comprises 5-enolpyruvylshikimate-3-phosphate synthase (2mEPSPS). In another embodiment, the plural of signature peptides comprises at least one sequence selected from the group consisting of SEQ ID NOs: 2-25. In another embodiment, the plural of signature peptides comprises at least two sequences selected from the group consisting of SEQ ID NOs: 2-25. In another embodiment, the plural of signature peptides comprises at least three sequences selected from the group consisting of SEQ ID NOs: 2-25. In another embodiment, the plural of signature peptides comprises SEQ ID NOs 3, 12, and 21. In another embodiment, the plural of signature peptides consist of SEQ ID NOs 3, 12, and 21.

In another embodiment, the one or more protein of interest comprises an aryloxyalkanoate dioxygenase (AAD). In another embodiment, the one or more protein of interest comprises aryloxyalkanoate dioxygenase-12 (AAD-12). In another embodiment, the plural of signature peptides comprises at least one sequence selected from the group consisting of SEQ ID NOs: 27-45. In another embodiment, the plural of signature peptides comprises at least two sequences selected from the group consisting of SEQ ID NOs: 27-45. In another embodiment, the plural of signature peptides comprises at least three sequences selected from the group consisting of SEQ ID NOs: 27-45. In another embodiment, the plural of signature peptides comprises SEQ ID NOs 28, 29, and 34. In another embodiment, the plural of signature peptides consist of SEQ ID NOs 28, 29, and 34.

In another embodiment, the one or more protein of interest comprises a bialaphos resistance (bar) gene product or phosphinothricin N-acetyltransferase (PAT) enzyme. In another embodiment, the one or more protein of interest comprises phosphinothricin acetyltransferase (PAT). In another embodiment, the plural of signature peptides comprises at least one sequence selected from the group consisting of SEQ ID NOs: 47-60. In another embodiment, the plural of signature peptides comprises at least two sequences selected from the group consisting of SEQ ID NOs: 47-60. In another embodiment, the plural of signature peptides comprises at least three sequences selected from the group consisting of SEQ ID NOs: 47-60. In another embodiment, the plural of signature peptides comprises SEQ ID NOs 49, 55, and 56. In another embodiment, the plural of signature peptides consist of SEQ ID NOs 49, 55, and 56.

In another aspect, provided is a high-throughput method of quantitating one or more protein of interest with known amino acid sequence in a plant-based sample. The method comprises using the system provided herein.

### BRIEF DESCRIPTION OF THE FIGURES

FIG. 1 shows a representative analysis work flow for the methods and systems disclosed herein.

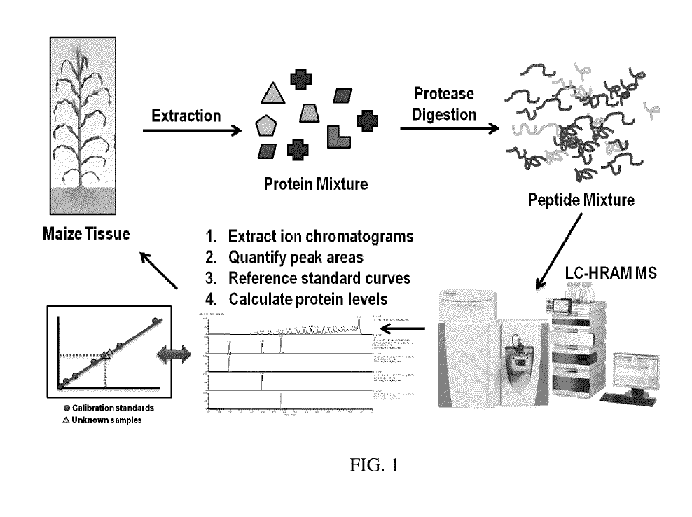

FIG. 2 shows another representative data from HRAM LC-MS for Standard Chromatogram 500 ng/mL Synthetic Peptide: total ion current (first panel from the top); combined extracted ion (second panel from the top); extracted ion 367.2082 m/z-EISGTVK (2+) (third panel from the top or the middle panel); extracted ion 367.1850 m/z-DVASWR (2+) (second panel from the bottom); and extracted ion 484.7798 m/z-VNGIGGLPGGK (2+) (first panel from the bottom). Extracted window is 2.0 ppm for all ions.

FIG. 3 shows representative data from HRAM LC-MS for Trypsin Digested Transgenic Soybean Sample Chromatogram: total ion current (first panel from the top); combined extracted ion (second panel from the top); extracted ion 367.2082 m/z-EISGTVK (2+) (third panel from the top or the middle panel); extracted ion 367.1850 m/z-DVASWR (2+) (second panel from the bottom); and extracted ion 484.7798 m/z-VNGIGGLPGGK (2+) (first panel from the bottom). Extracted window is 2.0 ppm for all ions.

FIG. 4 shows representative date of stacked HRAM LC-MS Standard (upper panel) and Transgenic (lower panel) Extracted Ion Chromatograms with peptide annotation for quantitation. Extracted window is 2.0 ppm for all ions.

FIG. 5 shows another representative data from HRAM LC-MS for Standard Chromatogram 500 ng/mL Synthetic Peptide: total ion current (first panel from the top); combined extracted ion (second panel from the top); extracted ion 346.6889 m/z-FGAIER (2+) (third panel from the top or the middle panel); extracted ion 621.8563 m/z-IGGGDIVAISNVK (2+) (second panel from the bottom); and extracted ion 598.2831 m/z-AAYDALDEATR (2+) (first panel from the bottom). Extracted window is 2.0 ppm for all ions.

FIG. 6 shows representative data from HRAM LC-MS for Trypsin Digested Transgenic Soybean Sample Chromatogram: total ion current (first panel from the top); combined extracted ion (second panel from the top); extracted ion 346.6889 m/z-FGAIER (2+) (third panel from the top or the middle panel); extracted ion 621.8563 m/z-IGGGDIVAISNVK (2+) (second panel from the bottom); and extracted ion 598.2831 m/z-AAYDALDEATR (2+) (first panel from the bottom). Extracted window is 2.0 ppm for all ions.

FIG. 7 shows representative date of stacked HRAM LC-MS Standard (upper panel) and Transgenic (lower panel) Extracted Ion Chromatograms with peptide annotation for quantitation. Extracted window is 2.0 ppm for all ions

FIG. 8 shows another representative data from HRAM LC-MS for Standard Chromatogram 500 ng/mL Synthetic Peptide: total ion current (first panel from the top); combined extracted ion (second panel from the top); extracted ion 928.9367 m/z-TEPQTPQEWIDDLER (2+) (third panel from the top or the middle panel); extracted ion 761.9330 m/z-SVVAVIGLPNDPSVR (2+) (second panel from the bottom); and extracted ion 565.8013 m/z-LHEALGYTAR (2+) (first panel from the bottom). Extracted window is 2.0 ppm for all ions.

FIG. 9 shows representative data from HRAM LC-MS for Trypsin Digested Transgenic Soybean Sample Chromatogram: total ion current (first panel from the top); combined extracted ion (second panel from the top); extracted ion 928.9367 m/z-TEPQTPQEWIDDLER (2+) (third panel from the top or the middle panel); extracted ion 761.9330 m/z-SVVAVIGLPNDPSVR (2+) (second panel from the bottom); and extracted ion 565.8013 m/z-LHEALGYTAR (2+) (first panel from the bottom). Extracted window is 2.0 ppm for all ions.

FIG. 10 shows representative date of stacked HRAM LC-MS Standard (upper panel) and Transgenic (lower panel) Extracted Ion Chromatograms with peptide annotation for quantitation. Extracted window is 2.0 ppm for all ions.

### DETAILED DESCRIPTION OF THE INVENTION

This invention is based on the discovery that selected signature peptides from precursor proteins can generate sensitive quantitation during multiplex analysis with particular instrumentation. Specifically in one embodiment, a liquid chromatography coupled to high resolution accurate mass spectrometry (LC-HRAM MS) method to detect protein expression levels of 5-enolpyruvylshikimate-3-phosphate synthase (2mEPSPS). The methods and systems disclosed herein are capable of analyzing 2mEPSPS by itself or combined with additional proteins for a multiplexing assay for quantitative analysis in plant extracts. Specifically in another embodiment, a liquid chromatography coupled to high resolution accurate mass spectrometry (LC-HRAM MS) method to detect protein expression levels of aryloxyalkanoate dioxygenase-12 (AAD-12). The methods and systems disclosed herein are capable of analyzing AAD-12 by itself or combined with additional proteins for a multiplexing assay for quantitative analysis in plant extracts. Specifically in yet another embodiment, a liquid chromatography coupled to high resolution accurate mass spectrometry (LC-HRAM MS) method to detect protein expression levels of phosphinothricin acetyltransferase (PAT). The methods and systems disclosed herein are capable of analyzing PAT by itself or combined with additional proteins for a multiplexing assay for quantitative analysis in plant extracts.

It is of significance to have a sensitive multiplex assay that is capable of selectively detecting multiple transgenic proteins of interest due to increasing numbers of transgenic proteins being co-expressed or “stacked” to achieve tolerance to multiple herbicides or to provide multiple modes of action to insect resistance. Currently, all relevant technologies for transgenic protein expression detection rely heavily on traditional immunochemistry technologies which present a challenge to accommodate the volume of data required to generate per sample.

The mass spectrometry detection for quantitative studies is typically accomplished using selected reaction monitoring (SRM). Using particular type of instrumentation, initial mass-selection of ion of interest formed in the source, followed by, dissociation of this precursor (protein) ion in the collision region of the mass spectrometer (MS), then mass-selection, and counting, of a specific product (peptide) ion. In some embodiment, counts per unit time may provide an integratable peak area from which amounts or concentration of analytes can be determined. In some embodiment, the use of high resolution accurate mass (HRAM) monitoring for quantitation, performed on a HRAM capable mass spectrometer, may include, but is not limited to, hybrid quadrupole-time-of-flight, quadrupole-orbitrap, ion trap-orbitrap, or quadrupole-ion-trap-orbitrap (tribrid) mass spectrometers. Using particular type of instrumentation, peptides are not subject to fragmentation conditions, but rather are measured as intact peptides using full scan or targeted scan modes (for example selective ion monitoring mode or SIM). Integratable peak area can be determined by generating an extracted ion chromatogram for each specific analyte and amounts or concentration of analytes can be calculated. The high resolution and accurate mass nature of the data enable highly specific and sensitive ion signals for the analyte (protein and/or peptide) of interest.

Unless otherwise stated, the following terms used in this application, including the specification and claims, have the definitions given below. It must be noted that, as used in the specification and the appended claims, the singular forms “a,” “an,” and “the” include plural referents unless the context clearly dictates otherwise.

As used herein, the term “bioconfinement” refers to restriction of the movement of genetically modified plants or their genetic material to designated areas. The term includes physical, physicochemical, biological confinement, as well as other forms of confinement that prevent the survival, spread or reproduction of a genetically modified plants in the natural environment or in artificial growth conditions.

As used herein, the term “complex protein sample” is used to distinguish a sample from a purified protein sample. A complex protein sample contains multiple proteins, and may additionally contain other contaminants.

As used herein, the general term “mass spectrometry” or “MS” refers to any suitable mass spectrometry method, device or configuration including, e.g., electrospray ionization (ESI), matrix-assisted laser desorption/ionization (MALDI) MS, MALDI-time of flight (TOF) MS, atmospheric pressure (AP) MALDI MS, vacuum MALDI MS, or combinations thereof. Mass spectrometry devices measure the molecular mass of a molecule (as a function of the molecule's mass-to-charge ratio) by measuring the molecule's flight path through a set of magnetic and electric fields. The mass-to-charge ratio is a physical quantity that is widely used in the electrodynamics of charged particles. The mass-to-charge ratio of a particular peptide can be calculated, a priori, by one of skill in the art. Two particles with different mass-to-charge ratio will not move in the same path in a vacuum when subjected to the same electric and magnetic fields.

Mass spectrometry instruments consist of three modules: an ion source, which splits the sample molecules into ions; a mass analyzer, which sorts the ions by their masses by applying electromagnetic fields; and a detector, which measures the value of an indicator quantity and thus provides data for calculating the abundances of each ion present. The technique has both qualitative and quantitative applications. These include identifying unknown compounds, determining the isotopic composition of elements in a molecule, determining the structure of a compound by observing its fragmentation, and quantifying the amount of a compound in a sample.

A detailed overview of mass spectrometry methodologies and devices can be found in the following references which are hereby incorporated by reference: Can and Annan (1997) Overview of peptide and protein analysis by mass spectrometry. In: Current Protocols in Molecular Biology, edited by Ausubel, et al. New York: Wiley, p. 10.21.1-10.21.27; Paterson and Aebersold (1995) Electrophoresis 16: 1791-1814; Patterson (1998) Protein identification and characterization by mass spectrometry. In: Current Protocols in Molecular Biology, edited by Ausubel, et al. New York: Wiley, p. 10.22.1-10.22.24; and Domon and Aebersold (2006) Science 312(5771):212-17.

As the term is used herein, proteins and/or peptides are “multiplexed” when two or more proteins and/or peptides of interest are present in the same sample.

As used herein, a “plant trait” may refer to any single feature or quantifiable measurement of a plant.

As used herein, the phrase “peptide” or peptides” may refer to short polymers formed from the linking, in a defined order, of α-amino acids. Peptides may also be generated by the digestion of polypeptides, for example proteins, with a protease.

As used herein, the phrase “protein” or proteins” may refer to organic compounds made of amino acids arranged in a linear chain and joined together by peptide bonds between the carboxyl and amino groups of adjacent amino acid residues. The sequence of amino acids in a protein is defined by the sequence of a gene, which is encoded in the genetic code. In general, the genetic code specifies 20 standard amino acids, however in certain organisms the genetic code can include selenocysteine—and in certain archaea-pyrrolysine. The residues in a protein are often observed to be chemically modified by post-translational modification, which can happen either before the protein is used in the cell, or as part of control mechanisms. Protein residues may also be modified by design, according to techniques familiar to those of skill in the art. As used herein, the term “protein” encompasses linear chains comprising naturally occurring amino acids, synthetic amino acids, modified amino acids, or combinations of any or all of the above.

As used herein, the term “single injection” refers to the initial step in the operation of a MS or LC-MS device. When a protein sample is introduced into the device in a single injection, the entire sample is introduced in a single step.

As used herein, the phrase “signature peptide” refers an identifier (short peptide) sequence of a specific protein. Any protein may contain an average of between 10 and 100 signature peptides. Typically signature peptides have at least one of the following criteria: easily detected by mass spectroscopy, predictably and stably eluted from a liquid chromatography (LC) column, enriched by reversed phase high performance liquid chromatography (RP-HPLC), good ionization, good fragmentation, or combinations thereof. A peptide that is readily quantified by mass spectrometry typically has at least one of the following criteria: readily synthesized, ability to be highly purified (>97%), soluble in ≦20% acetonitrile, low non-specific binding, oxidation resistant, post-synthesis modification resistant, and a hydrophobicity or hydrophobicity index ≧10 and ≦40. The hydrophobicity index can be calculated according to Krokhin, Molecular and Cellular Proteomics 3 (2004) 908, which is incorporated by reference. It's known that a peptide having a hydrophobicity index less than 10 or greater than 40 may not be reproducibly resolved or eluted by a RP-HPLC column.

As used herein, the term “stacked” refers to the presence of multiple heterologous polynucleotides incorporated in the genome of a plant.

Tandem mass spectrometry: In tandem mass spectrometry, a parent ion generated from a molecule of interest may be filtered in a mass spectrometry instrument, and the parent ion subsequently fragmented to yield one or more daughter ions that are then analyzed (detected and/or quantified) in a second mass spectrometry procedure. In some embodiments, the use of tandem mass spectrometry is excluded. In these embodiments, tandem mass spectrometry is not used in the methods and systems provided. Thus, neither parent ions nor daughter ions are generated in these embodiments.

As used herein, the term “transgenic plant” includes reference to a plant which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenic plants initially so altered as well as those created by sexual crosses or asexual propagation from the initial transgenic plant.

Any plants that provide useful plant parts may be treated in the practice of the present invention. Examples include plants that provide flowers, fruits, vegetables, and grains.

As used herein, the phrase “plant” includes dicotyledonous plants and monocotyledonous plants. Examples of dicotyledonous plants include tobacco, Arabidopsis, soybean, tomato, papaya, canola, sunflower, cotton, alfalfa, potato, grapevine, pigeon pea, pea, Brassica, chickpea, sugar beet, rapeseed, watermelon, melon, pepper, peanut, pumpkin, radish, spinach, squash, broccoli, cabbage, carrot, cauliflower, celery, Chinese cabbage, cucumber, eggplant, and lettuce. Examples of monocotyledonous plants include corn, rice, wheat, sugarcane, barley, rye, sorghum, orchids, bamboo, banana, cattails, lilies, oat, onion, millet, and triticale. Examples of fruit include banana, pineapple, oranges, grapes, grapefruit, watermelon, melon, apples, peaches, pears, kiwifruit, mango, nectarines, guava, persimmon, avocado, lemon, fig, and berries. Examples of flowers include baby's breath, carnation, dahlia, daffodil, geranium, gerbera, lily, orchid, peony, Queen Anne's lace, rose, snapdragon, or other cut-flowers or ornamental flowers, potted-flowers, and flower bulbs.

The specificity allowed in a mass spectrometry approach for identifying a single protein from a complex sample is unique in that only the sequence of the protein of interest is required in order to identify the protein of interest. Compared to other formats of multiplexing, mass spectrometry is unique in being able to exploit the full length of a protein's primary amino acid sequence to target unique identifier-type portions of a protein's primary amino acid sequence to virtually eliminate non-specific detection. In some embodiments of the present invention, a proteolytic fragment or set of proteolytic fragments that uniquely identifies a protein(s) of interest is used to detect the protein(s) of interest in a complex protein sample.

In some embodiments, disclosed methods enable the quantification or determination of ratios of multiple proteins in a complex protein sample by a single mass spectrometry analysis, as opposed to measuring each protein of interest individually multiple times and compiling the individual results into one sample result.

In some embodiments, the present disclosure also provides methods useful for the development and use of transgenic plant technology. Specifically, disclosed methods may be used to maintain the genotype of transgenic plants through successive generations. Also, some embodiments of the methods disclosed herein may be used to provide high-throughput analysis of non-transgenic plants that are at risk of being contaminated with transgenes from neighboring plants, for example, by cross-pollination. By these embodiments, bioconfinement of transgenes may be facilitated and/or accomplished. In other embodiments, methods disclosed herein may be used to screen the results of a plant transformation procedure in a high-throughput manner to identify transformants that exhibit desirable expression characteristics

Any protein introduced into a plant via transgenic expression technology may be analyzed using methods of the invention. Proteins suitable for multiplex analysis according to the invention may confer an output trait that renders the transgenic plant superior to its nontransgenic counterpart. Non-limiting examples of desirable traits that may be conferred include herbicide resistance, resistance to insects, resistance to disease, resistance to environmental stress, enhanced yield, improved nutritional value, improved shelf life, altered oil content, altered oil composition, altered sugar content, altered starch content, production of plant-based pharmaceuticals, production of industrial products (for example polyhydroxyalkanoates: macromolecule polyesters considered ideal for replacing petroleum-derived plastics), and potential for bioremediation. Moreover, the expression of one or more transgenic proteins within a single plant species may be analyzed using methods of the present disclosure. The addition or modulation of two or more genes or desired traits into a single species of interest is known as gene stacking. Furthermore, the expression of one or more transgenic proteins may be analyzed concurrently in the presently disclosed multiplex analyses with one or more endogenous plant proteins.

Particularly suitable proteins that are expressed in transgenic plants are those that confer tolerance to herbicides for example the gene of 5′-enolpyruvyl-3′-phosphoshikimate synthase (EPSPS) or any variant thereof for conferring tolerance to glyphosate herbicides, the aryloxyalkanoate dioxygenase (AAD) for conferring tolerance to 2,4-D herbicides, the phosphinothricin acetyltransferase (PAT) for conferring tolerance to glufosinate herbicides, or combinations thereof.

The mass-to-charge ratio may be determined using a quadrupole analyzer. For example, in a “quadrupole” or “quadrupole ion trap” instrument, ions in an oscillating radio frequency field experience a force proportional to the DC potential applied between electrodes, the amplitude of the RF signal, and m/z. The voltage and amplitude can be selected so that only ions having a particular m/z travel the length of the quadrupole, while all other ions are deflected. Thus, quadrupole instruments can act as a “mass filter” and “mass detector” for the ions injected into the instrument.

Collision-induced dissociation (“CID”) is often used to generate the daughter ions for further detection. In CID, parent ions gain energy through collisions with an inert gas, such as argon, and subsequently fragmented by a process referred to as “unimolecular decomposition.” Sufficient energy must be deposited in the parent ion so that certain bonds within the ion can be broken due to increased energy.

The mass spectrometer typically provides the user with an ion scan; that is, the relative abundance of each m/z over a given range (for example 10 to 1200 amu). The results of an analyte assay, that is, a mass spectrum, can be related to the amount of the analyte in the original sample by numerous methods known in the art. For example, given that sampling and analysis parameters are carefully controlled, the relative abundance of a given ion can be compared to a table that converts that relative abundance to an absolute amount of the original molecule. Alternatively, molecular standards (e.g., internal standards and external standards) can be run with the samples and a standard curve constructed based on ions generated from those standards. Using such a standard curve, the relative abundance of a given ion can be converted into an absolute amount of the original molecule. Numerous other methods for relating the presence or amount of an ion to the presence or amount of the original molecule are well known to those of ordinary skill in the art.

The choice of ionization method can be determined based on the analyte to be measured, type of sample, the type of detector, the choice of positive versus negative mode, etc. Ions can be produced using a variety of methods including, but not limited to, electron ionization, chemical ionization, fast atom bombardment, field desorption, and matrix-assisted laser desorption ionization (MALDI), surface enhanced laser desorption ionization (SELDI), desorption electrospray ionization (DESI), photon ionization, electrospray ionization, and inductively coupled plasma. Electrospray ionization refers to methods in which a solution is passed along a short length of capillary tube, to the end of which is applied a high positive or negative electric potential. Solution reaching the end of the tube, is vaporized (nebulized) into a jet or spray of very small droplets of solution in solvent vapor. This mist of droplets flows through an evaporation chamber which is heated to prevent condensation and to evaporate solvent. As the droplets get smaller the electrical surface charge density increases until such time that the natural repulsion between like charges causes ions as well as neutral molecules to be released.

The effluent of an LC may be injected directly and automatically (i.e., “in-line”) into the electrospray device. In some embodiments, proteins contained in an LC effluent are first ionized by electrospray into a parent ion.

Various different mass analyzers can be used in liquid chromatography-mass spectrometry combination (LC-MS). Exemplary mass analyzers include, but not limited to, single quadrupole, triple quadrupole, ion trap, TOF (time of flight), and quadrupole-time of flight (Q-TOF).

The quadrupole mass analyzer may consist of 4 circular rods, set parallel to each other. In a quadrupole mass spectrometer (QMS), the quadrupole is the component of the instrument responsible for filtering sample ions, based on their mass-to-charge ratio (m/z). Ions are separated in a quadrupole based on the stability of their trajectories in the oscillating electric fields that are applied to the rods.

An ion trap is a combination of electric or magnetic fields that captures ions in a region of a vacuum system or tube. Ion traps can be used in mass spectrometry while the ion's quantum state is manipulated.

Time-of-flight mass spectrometry (TOFMS) is a method of mass spectrometry in which an ion's mass-to-charge ratio is determined via a time measurement. Ions are accelerated by an electric field of known strength. This acceleration results in an ion having the same kinetic energy as any other ion that has the same charge. The velocity of the ion depends on the mass-to-charge ratio. The time that it subsequently takes for the particle to reach a detector at a known distance is measured. This time will depend on the mass-to-charge ratio of the particle (heavier particles reach lower speeds). From this time and the known experimental parameters one can find the mass-to-charge ratio of the ion.

In some embodiments, the particular instrument used by the methods and/or systems provided may comprise a high fragmentation mode and a low fragmentation mode (or alternatively a non-fragmentation mode). Such different modes may include alternating scan high and low energy acquisition methodology to generate high resolution mass data. In some embodiments, the high resolution mass data may comprise a product data set (for example data derived from product ion (fragmented ions) under the high fragmentation mode) and a precursor data set (for example data derived from precursor ions (unfragmented ions) under the low fragmentation or non-fragmentation mode).

In some embodiments, the methods and/or systems provided use a mass spectrometer comprising a filtering device that may be used in the selection step, a fragmentation device that may be used in the fragmentation step, and/or one or more mass analyzers that may be used in the acquisition and/or mass spectrum creation step or steps.

The filtering device and/or mass analyzer may comprise a quadrupole. The selection step and/or acquisition step and/or mass spectrum creation step or steps may involve the use of a resolving quadrupole. Additionally or alternatively, the filtering device may comprise a two dimensional or three dimensional ion trap or time-of-flight (ToF) mass analyzer. The mass analyzer or mass analyzers may comprise or further comprise one or more of a time-of-flight mass analyzer and/or an ion cyclotron resonance mass analyzer and/or an orbitrap mass analyzer and/or a two dimensional or three dimensional ion trap.

Filtering by means of selection based upon mass-to-charge ratio (m/z) can be achieved by using a mass analyzer which can select ions based upon m/z, for example a quadrupole; or to transmit a wide m/z range, separate ions according to their m/z, and then select the ions of interest by means of their m/z value. An example of the latter would be a time-of-flight mass analyzer combined with a timed ion selector(s). The methods and/or systems provided may comprise isolating and/or separating the one or more proteins of interest, for example from two or more of a plurality of proteins, using a chromatographic technique for example liquid chromatography (LC). The method may further comprise measuring an elution time for the protein of interest and/or comparing the measured elution time with an expected elution time.

Additionally or alternatively, the proteins of interest may be separated using an ion mobility technique, which may be carried out using an ion mobility cell. Additionally, the proteins of interest may be selected by order or time of ion mobility drift. The method may further comprise measuring a drift time for the proteins of interest and/or comparing the measured drift time with an expected drift time.

In some embodiments, the methods and/or systems provided are label-free, where quantitation can be achieved by comparison of the peak intensity, or area under the mass spectral peak for the precursor or product m/z values of interest between injections and across samples. In some embodiments, internal standard normalization may be used to account for any known associated analytical error. Another label-free method of quantification, spectral counting, involves summing the number of fragment ion spectra, or scans, that are acquired for each given peptide, in a non-redundant or redundant fashion. The associated peptide mass spectra for each protein are then summed, providing a measure of the number of scans per protein with this being proportional to its abundance. Comparison can then be made between samples/injections.

In some embodiments, the ion source is selected from the group consisting of: (1) an electrospray ionization (“ESI”) ion source; (2) an atmospheric pressure photo ionization (“APPI”) ion source; (3) an atmospheric pressure chemical ionization (“APCI”) ion source; (4) a matrix assisted laser desorption ionization (“MALDI”) ion source; (5) a laser desorption ionization (“LDI”) ion source; (6) an atmospheric pressure ionization (“API”) ion source; (7) a desorption ionization on silicon (“DIOS”) ion source; (8) an electron impact (“EI”) ion source; (9) a chemical ionization (“CI”) ion source; (10) a field ionization (“FI”) ion source; (11) a field desorption (“FD”) ion source; (12) an inductively coupled plasma (“ICP”) ion source; (13) a fast atom bombardment (“FAB”) ion source; (14) a liquid secondary ion mass spectrometry (“LSIMS”) ion source; (15) a desorption electrospray ionization (“DESI”) ion source; (16) a nickel-63 radioactive ion source; (17) an atmospheric pressure matrix assisted laser desorption ionization ion source; and (18) a thermospray ion source.

In some embodiments, the methods and/or systems provided comprise an apparatus and/or control system configured to execute a computer program element comprising computer readable program code means for causing a processor to execute a procedure to implement the methods.

In some embodiments, the methods and/or systems provided use an alternating low and elevated energy scan function in combination with liquid chromatography separation of a plant extract. A list of information for proteins of interest can be provided including, but is not limited to, m/z of precursor ion, m/z of product ions, retention time, ion mobility drift time and rate of change of mobility. During the course of the LC separation and as the target ions elute into the mass spectrometer (and as either low energy precursor ions, or elevated energy product ions are detected, or the retention time window is activated) the mass analyzer of the methods and/or systems provided may select a narrow m/z range (of a variable and changeable width) to pass ions through to the gas cell. Accordingly, the signal to noise ratio can be enhanced significantly for quantification of proteins of interest.

In some embodiments, at a chromatographic retention time when a targeted protein of interest is about to elute into the mass spectrometer ion source, the mass analyzer of the methods and/or systems provided can select a narrow m/z range (of a variable and changeable width) according to the targeted precursor ion. These selected ions are then transferred to an instrument stage capable of dissociating the ions by means of alternate and repeated switches between a high fragmentation mode where the sample precursor ions are substantially fragmented into product ions and a low fragmentation mode (or non-fragmentation mode) where the sample precursor ions are not substantially fragmented. Typically high resolution, accurate mass spectra are acquired in both modes and at the end of the experiment associated precursor and product ions are recognized by the closeness in fit of their chromatographic elution times and optionally other physicochemical properties. The signal intensity of either the precursor ion or the product ion associated with targeted proteins of interest can be used to determine the quantity of the proteins in the plant extract.

Those skilled in the art would understand certain variation can exist based on the disclosure provided. Thus, the following examples are given for the purpose of illustrating the invention and shall not be construed as being a limitation on the scope of the invention or claims.

### EXAMPLES

### Example 1

Plant samples (for example grain, leaf, root, forage, pollen) are extracted with assay buffer PBST combined with dithiothreitol (DTT). SEQ ID NO: 1 provides the protein sequence of 5-enolpyruvylshikimate-3-phosphate synthase (2mEPSPS):

MAGAEEIVLQPIKEISGTVKLPGSKSLSNRILLLAALSEGTTVVDNLLNSE
DVHYMLGALRTLGLSVEADKAAKRAVVVGCGGKFPVEDAKEEVQLFLGNAG
IAMRSLTAAVTAAGGNATYVLDGVPRMRERPIGDLVVGLKQLGADVDCFLG
TDCPPVRVNGIGGLPGGKVKLSGSISSQYLSALLMAAPLALGDVEIEIIDK
LISIPYVEMTLRLMERFGVKAEHSDSWDRFYIKGGQKYKSPKNAYVEGDAS
SASYFLAGAAITGGTVTVEGCGTTSLQGDVKFAEVLEMMGAKVTWTETSVT
VTGPPREPFGRKHLKAIDVNMNKMPDVAMTLAVVALFADGPTAIRDVASWR
VKETERMVAIRTELTKLGASVEEGPDYCIITPPEKLNVTAIDTYDDHRMAM
AFSLAACAEVPVTIRDPGCTRKTFPDYFDVLSTFVKN.

TABLE 1
Candidate signature peptides for 5-
enolpyruvylshikimate-3-phosphate synthase
(2mEPSPS)

| | |
|---|---|
| SEQ ID NO: 2 | AGAEEIVLQPIK |
| SEQ ID NO: 3 | EISGTVK |
| SEQ ID NO: 4 | ILLLAALSEGTTVVDNLLNSEDVHYMKGALR |
| SEQ ID NO: 5 | TLGLSVEADK |
| SEQ ID NO: 6 | AVVVGCGGK |
| SEQ ID NO: 7 | FPVEDAK |
| SEQ ID NO: 8 | EEVQLFLGNAGIAMR |
| SEQ ID NO: 9 | SLTAAVTAAGGNATYVLDGVPR |
| SEQ ID NO: 10 | ERPIGDLVVGLK |
| SEQ ID NO: 11 | QLGADVDCFLGTDCPPVR |
| SEQ ID NO: 12 | VNGIGGLPGGK |
| SEQ ID NO: 13 | LSGSISSQYLSALLMAAPLALGDVEIEIIDK |
| SEQ ID NO: 14 | LISIPYVEMTLR |
| SEQ ID NO: 15 | AEHSDSWDR |
| SEQ ID NO: 16 | NAYVEGDASSASYFLAGAAITGGTVTVEGCGTTSLQGD |
| | VK |
| SEQ ID NO: 17 | FAEVLEMMGAK |
| SEQ ID NO: 18 | VTWTETSVTVTGPPR |
| SEQ ID NO: 19 | AIDVNMNK |
| SEQ ID NO: 20 | MPDVAMTLAVVALFADGPTAIR |
| SEQ ID NO: 21 | DVASWR |
| SEQ ID NO: 22 | LGASVEEGPDYCIITPPEK |
| SEQ ID NO: 23 | LNVTAIDTYDDHR |
| SEQ ID NO: 24 | MAMAFSLAACAEVPVTIR |
| SEQ ID NO: 25 | TFPDYFDVLSTFVK |

The extracted proteins are denatured and then proteolytically digested by adding trypsin protease and incubating at 37° C. for 15-20 hours. The digestion reactions are then acidified with formic acid (pH=1-2) and are analyzed using LC-MS. The protein sequence for 2mEPSPS is analyzed and digested in silico to generate theoretical peptide fragments to be detected and measured by LC-MS. Candidate signature peptides for 5-enolpyruvylshikimate-3-phosphate synthase (2mEPSPS) after trypsin digestion are listed in Table 1.

Surprisingly three candidate signature peptides provide good correlation for quantitation using LC-MS, as compared to results from Enzyme-Linked Immunosorbent Assay (ELISA) or other quantitation methods. These three signature peptides are EISGTVK (SEQ ID NO: 3), DVASWR (SEQ ID NO: 21), and VNGIGGLPGGK (SEQ ID NO: 12). Both commercially synthesized peptides of these sequences as well as microbial-derived 2mEPSPS protein are used as analytical reference standards through the same digestion process as described above, where synthetic peptides can directly serve as an analytical reference standard for protein quantitation.

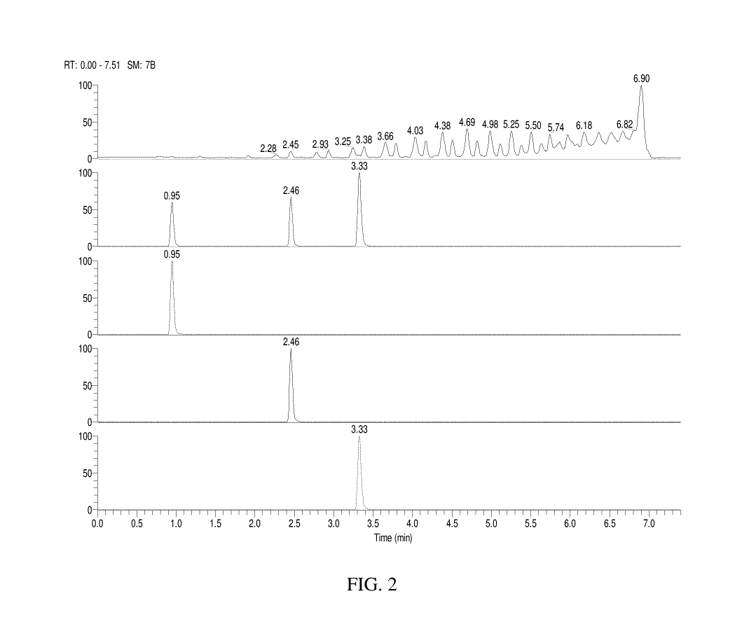

Representative data from HRAM LC-MS for Standard Chromatogram 500 ng/mL Synthetic Peptide are shown in FIG. 2, where comparison among total ion current (first panel from the top); combined extracted ion (second panel from the top); extracted ion 367.2082 m/z-EISGTVK (2+) (third panel from the top or the middle panel); extracted ion 367.1850 m/z-DVASWR (2+) (second panel from the bottom); and extracted ion 484.7798 m/z-VNGIGGLPGGK (2+) (first panel from the bottom) can identify signature peak(s) for each signature peptide. Extracted window is 2.0 ppm for all ions.

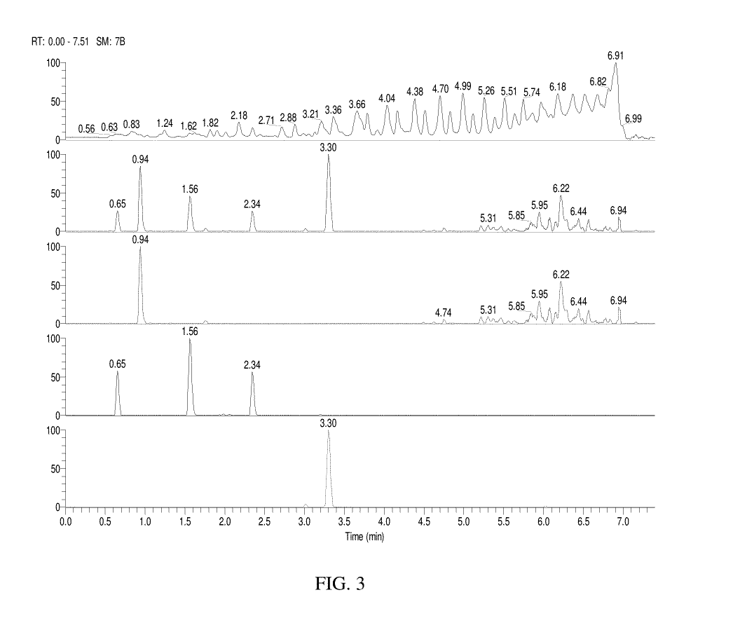

Another representative data from HRAM LC-MS for Trypsin Digested Transgenic Soybean Sample Chromatogram are shown in FIG. 3, where comparison among total ion current (first panel from the top); combined extracted ion (second panel from the top); extracted ion 367.2082 m/z-EISGTVK (2+) (third panel from the top or the middle panel); extracted ion 367.1850 m/z-DVASWR (2+) (second panel from the bottom); and extracted ion 484.7798 m/z-VNGIGGLPGGK (2+) (first panel from the bottom) can also identify signature peak(s) for each signature peptide. Extracted window is 2.0 ppm for all ions.

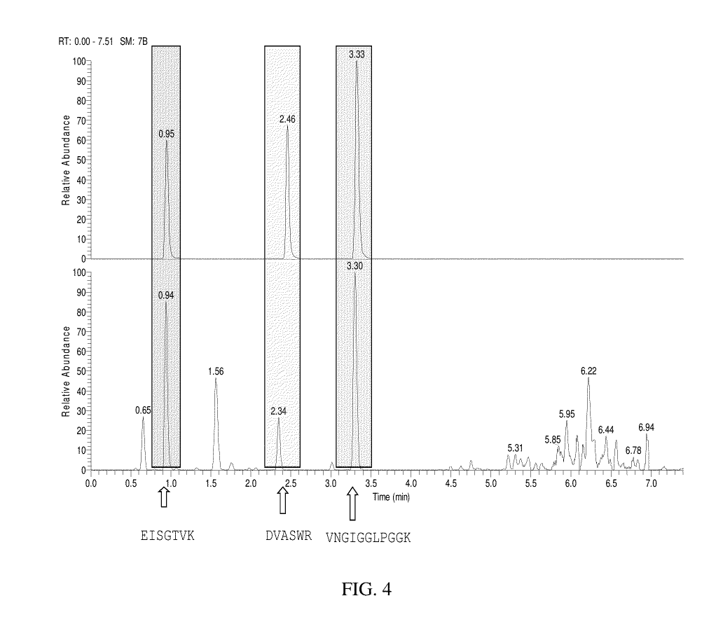

Peak area of signature peak(s) for each signature peptide can be calculated and representative date of stacked HRAM LC-MS Standard (upper panel) and Transgenic (lower panel) Extracted Ion Chromatograms with peptide annotation are shown in FIG. 4 for quantitation. Extracted window is 2.0 ppm for all ions.

### Example 2

Plant samples (for example grain, leaf, root, forage, pollen) are extracted with assay buffer PBST combined with dithiothreitol (DTT). The extracted proteins are denatured and then proteolytically digested by adding trypsin protease and incubating at 37° C. for 15-20 hours. The digestion reactions are then acidified with formic acid (pH=1-2) and are analyzed using LC-MS. SEQ ID NO: 26 provides the AAD-12 Protein Sequence:

MAQTTLQITPTGATLGATVTGVHLATLDDAGFAALHAAWLQHALLIFPGQH
LSNDQQITFAKRFGAIERIGGGDIVAISNVKADGTVRQHSPAEWDDMMKVI
VGNMAWHADSTYMPVMAQGAVFSAEVVPAVGGRTCFADMRAAYDALDEATR
ALVHQRSARHSLVYSQSKLGHVQQAGSAYIGYGMDTTATPLRPLVKVHPET
GRPSLLIGRHAHAIPGMDAAESERFLEGLVDWACQAPRVHAHQWAAGDVVV
WDNRCLLHRAEPWDFKLPRVMWHSRLAGRPETEGAALV.

TABLE 2
Candidate signature peptides for aryloxyalkanoate
dioxygenase-12 (AAD-12)

| | |
|---|---|
| SEQ ID NO: 27 | MAQTTLQITPTGATLLGATVTGVHLATLDDAGFAALHA |
| | AWLQHALLIFPGQHLSNDQQITFAK |
| SEQ ID NO: 28 | FGAIER |
| SEQ ID NO: 29 | IGGGDIVAISNVK |
| SEQ ID NO: 30 | ADGTVR |
| SEQ ID NO: 31 | QHSPAEWDDMMK |
| SEQ ID NO: 32 | VIVGNMAWHADSTYMPVMAQGAVFSAEVVPAVGGR |
| SEQ ID NO: 33 | TCFADMR |
| SEQ ID NO: 34 | AAYDALDEATR |
| SEQ ID NO: 35 | ALVHQR |
| SEQ ID NO: 36 | HSLVYSQSK |
| SEQ ID NO: 37 | LQHVQQAGSAYIGYGMDTTATPLRPLVK |
| SEQ ID NO: 38 | VHPETGRPSLLIGR |
| SEQ ID NO: 39 | HAHAIPGMDAAESER |
| SEQ ID NO: 40 | FLEGLVDWACQAPR |
| SEQ ID NO: 41 | VHAHQWAAGDVVVWDNR |
| SEQ ID NO: 42 | CLLHR |
| SEQ ID NO: 43 | AEPWDFK |
| SEQ ID NO: 44 | VMWHSR |
| SEQ ID NO: 45 | LAGRPETEGAALV |

The protein sequence for AAD-12 is analyzed and digested in silico to generate theoretical peptide fragments to be detected and measured by LC-MS. Candidate signature peptides for AAD-12 after trypsin digestion are listed in Table 2.

Surprisingly three candidate signature peptides provide good correlation for quantitation using LC-MS, as compared to results from Enzyme-Linked Immunosorbent Assay (ELISA) or other quantitation methods. These three signature peptides are FGAIER (SEQ ID NO: 28), IGGGDIVAISNVK (SEQ ID NO: 29), and AAYDALDEATR (SEQ ID NO: 34). Both commercially synthesized peptides of these sequences as well as microbial-derived AAD-12 protein are used as analytical reference standards through the same digestion process as described above, where synthetic peptides can directly serve as an analytical reference standard for protein quantitation.

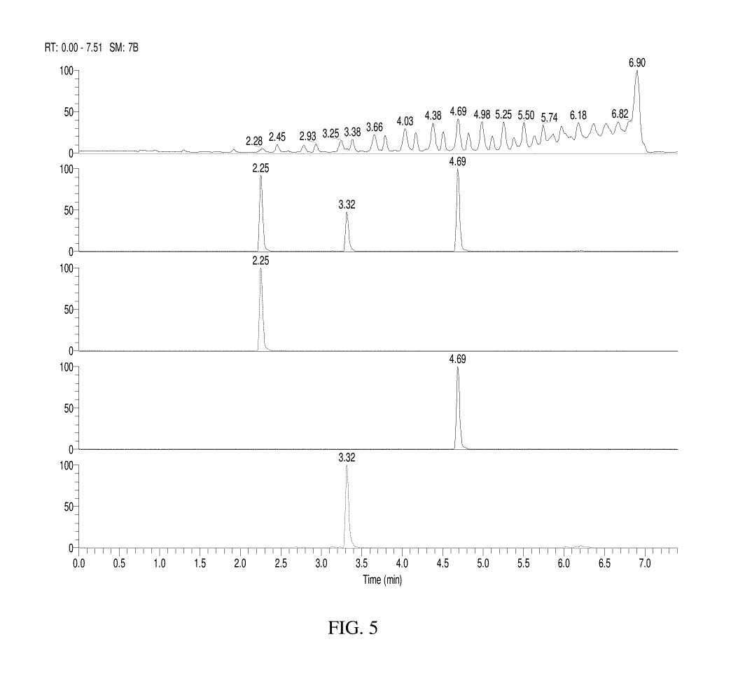

Representative data from HRAM LC-MS for Standard Chromatogram 500 ng/mL Synthetic Peptide are shown in FIG. 5, where comparison among total ion current (first panel from the top); combined extracted ion (second panel from the top); extracted ion 346.6889 m/z-FGAIER (2+) (third panel from the top or the middle panel); extracted ion 621.8563 m/z-IGGGDIVAISNVK (2+) (second panel from the bottom); and extracted ion 598.2831 m/z-AAYDALDEATR (2+) (first panel from the bottom). Extracted window is 2.0 ppm for all ions.

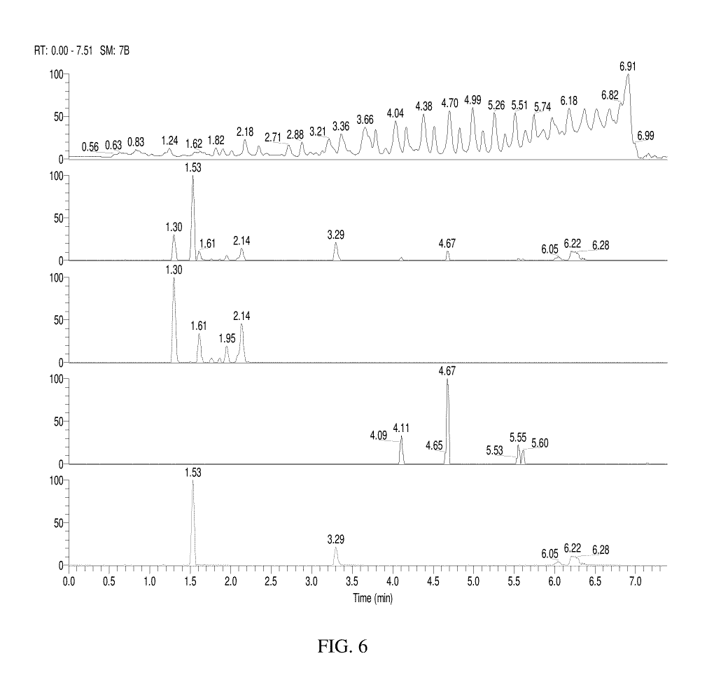

Another representative data from HRAM LC-MS for Trypsin Digested Transgenic Soybean Sample Chromatogram are shown in FIG. 6, where comparison among total ion current (first panel from the top); combined extracted ion (second panel from the top); extracted ion 346.6889 m/z-FGAIER (2+) (third panel from the top or the middle panel); extracted ion 621.8563 m/z-IGGGDIVAISNVK (2+) (second panel from the bottom); and extracted ion 598.2831 m/z-AAYDALDEATR (2+) (first panel from the bottom) can also identify signature peak(s) for each signature peptide. Extracted window is 2.0 ppm for all ions.

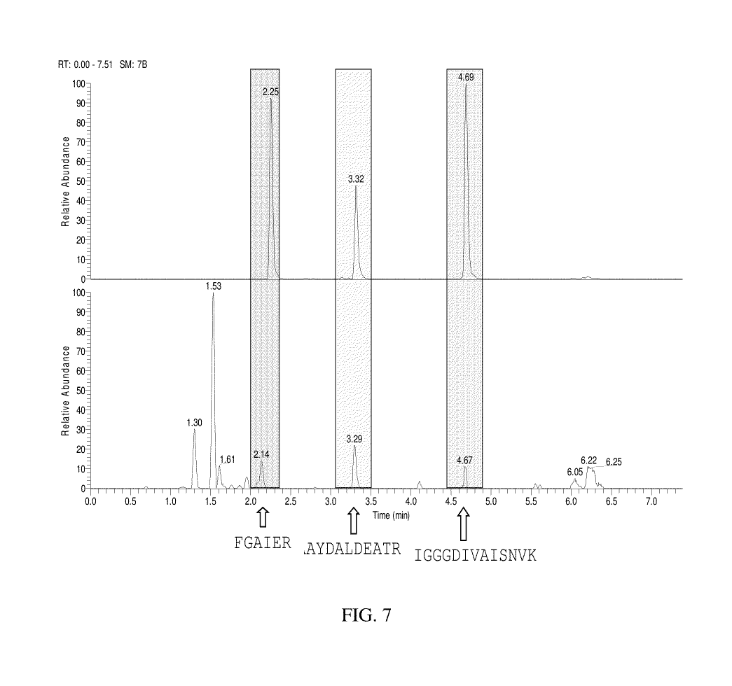

Peak area of signature peak(s) for each signature peptide can be calculated and representative date of stacked HRAM LC-MS Standard (upper panel) and Transgenic (lower panel) Extracted Ion Chromatograms with peptide annotation are shown in FIG. 7 for quantitation. Extracted window is 2.0 ppm for all ions.

### Example 3

Plant samples (for example grain, leaf, root, forage, pollen) are extracted with assay buffer PBST combined with dithiothreitol (DTT). The extracted proteins are denatured and then proteolytically digested by adding trypsin protease and incubating at 37° C. for 15-20 hours. The digestion reactions are then acidified with formic acid (pH=1-2) and are analyzed using LC-MS. SEQ ID NO: 46 provides the protein sequence of phosphinothricin acetyltransferase (PAT):

MSPERRPVEIRPATAADMAAVCDIVNHYIETSTVNPRTEPQTPQEWIDDLE
RLQDRYPWLVAEVEGVVAGIAYAGPWKARNAYDWTVESTVYVSHRHQRLGL
GSTLYTHLLKSMEAQGFKSVVAVIGLPNDPSVRLHEALGYTARGTLRAAGY
KHGGWHDVGFWQRDFELPAPPRPVRPVTQI.

The protein sequence for PAT is analyzed and digested in silico to generate theoretical peptide fragments to be detected and measured by LC-MS. Candidate signature peptides for phosphinothricin acetyltransferase (PAT) after trypsin digestion are listed in Table 3.

TABLE 3
Candidate signature peptides for phosphinothricin
acetyltransferase (PAT)

| | |
|---|---|
| SEQ ID NO: 47 | MSPER |
| SEQ ID NO: 48 | RPVEIRPATAADMAAVCDIVNHYIETSTVNFR |
| SEQ ID NO: 49 | TEPQTPQEWIDDLER |
| SEQ ID NO: 50 | LQDR |
| SEQ ID NO: 51 | YPWLVAEVEGVVAGIAYAGPWK |
| SEQ ID NO: 52 | NAYDWTVESTVYVSHR |
| SEQ ID NO: 53 | LGLGSTLYTHLLK |
| SEQ ID NO: 54 | SMEAQGFK |
| SEQ ID NO: 55 | SVVAVIGLPNDPSVR |
| SEQ ID NO: 56 | LHEALGYTAR |
| SEQ ID NO: 57 | GTLR |
| SEQ ID NO: 58 | AAGYK |
| SEQ ID NO: 59 | HGGWHDVGFWQR |
| SEQ ID NO: 60 | DFELPAPPRPVRPVTQI |

Surprisingly three candidate signature peptides provide good correlation for quantitation using LC-MS, as compared to results from Enzyme-Linked Immunosorbent Assay (ELISA) or other quantitation methods. These three signature peptides are TEPQTPQEWIDDLER (SEQ ID NO: 49), SVVAVIGLPNDPSVR (SEQ ID NO: 55), and LHEALGYTAR (SEQ ID NO: 56). Both commercially synthesized peptides of these sequences as well as microbial-derived PAT protein are used as analytical reference standards through the same digestion process as described above, where synthetic peptides can directly serve as an analytical reference standard for protein quantitation.

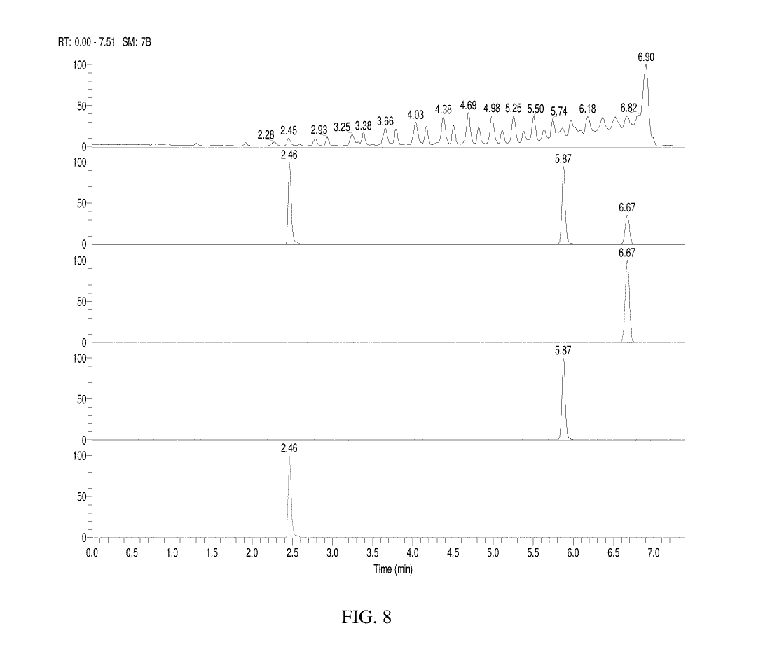

Representative data from HRAM LC-MS for Standard Chromatogram 500 ng/mL Synthetic Peptide are shown in FIG. 8, where comparison among total ion current (first panel from the top); combined extracted ion (second panel from the top); extracted ion 928.9367 m/z-TEPQTPQEWIDDLER (2+) (third panel from the top or the middle panel); extracted ion 761.9330 m/z-SVVAVIGLPNDPSVR (2+) (second panel from the bottom); and extracted ion 565.8013 m/z-LHEALGYTAR (2+) (first panel from the bottom) can identify signature peak(s) for each signature peptide. Extracted window is 2.0 ppm for all ions.

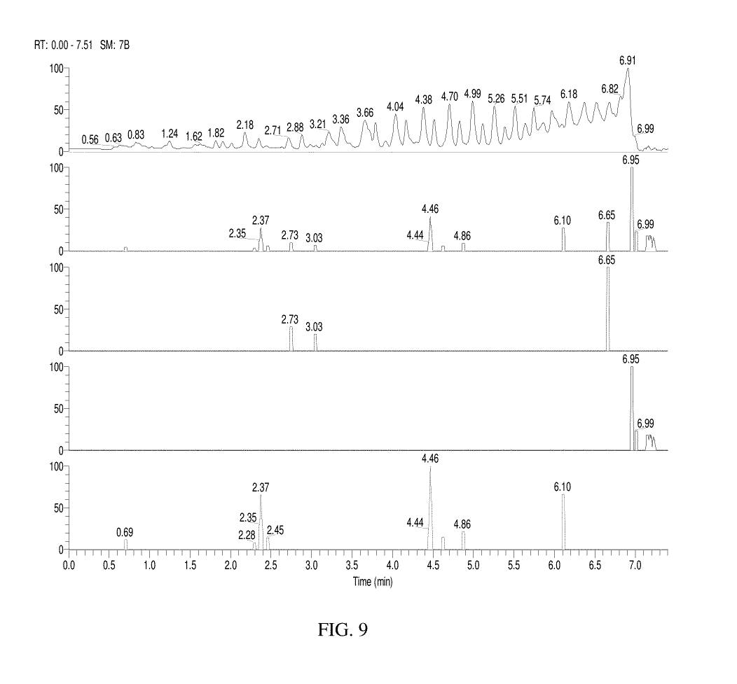

Another representative data from HRAM LC-MS for Trypsin Digested Transgenic Soybean Sample Chromatogram are shown in FIG. 9, where comparison among total ion current (first panel from the top); combined extracted ion (second panel from the top); extracted ion 928.9367 m/z-TEPQTPQEWIDDLER (2+) (third panel from the top or the middle panel); extracted ion 761.9330 m/z-SVVAVIGLPNDPSVR (2+) (second panel from the bottom); and extracted ion 565.8013 m/z-LHEALGYTAR (2+) (first panel from the bottom) can also identify signature peak(s) for each signature peptide. Extracted window is 2.0 ppm for all ions.

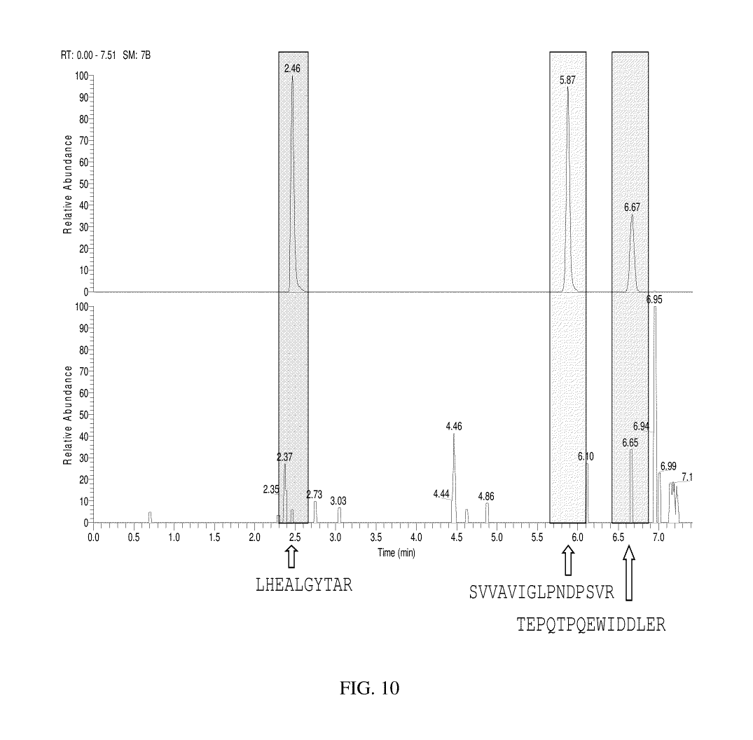

Peak area of signature peak(s) for each signature peptide can be calculated and representative date of stacked HRAM LC-MS Standard (upper panel) and Transgenic (lower panel) Extracted Ion Chromatograms with peptide annotation are shown in FIG. 10 for quantitation. Extracted window is 2.0 ppm for all ions.

We claim:

1. A high-throughput method of quantitating one or more protein of interest with known amino acid sequence in a plant-based sample, the method comprising:
(a) extracting proteins from a plant-based sample;
(b) digesting proteins extracted from step (a) to obtain peptides;
(c) separating the peptides in a single step;
(d) determining a plural of signature peptides from the protein of interest with known amino acid sequence;
(e) measuring the plural of signature peptides using high resolution accurate mass spectrometry (HRAM MS); and
(f) quantitating the protein of interest with known amino acid sequence based on measurements of the signature peptides.

2. The method of claim 1, wherein the peptides are separated in a single step by column chromatography.

3. The method of claim 1, wherein mass spectral data for the peptides corresponding to the protein of interest are obtained in a single step.

4. The method of claim 1, wherein the plant-based sample is from a transgenic plant.

5. The method of claim 1, wherein the one or more protein of interest comprises 5-enolpyruvylshikimate-3-phosphate synthase (2mEPSPS), aryloxyalkanoate dioxygenase-12 (AAD-12), and/or phosphinothricin acetyltransferase (PAT).

6. The method of claim 1, wherein the plural of signature peptides comprises at least three sequences selected from the group consisting of SEQ ID NOs: 2-25, 27-45, and 47-60.

7. The method of claim 1, wherein the plural of signature peptides comprises (1) SEQ ID NOs 3, 12, and 21; (2) SEQ ID NOs 28, 29, and 34; and/or (3) SEQ IN NOs: 49, 55, and 56.

8. The method of claim 1, wherein the plural of signature peptides consist of (1) SEQ ID NOs 3, 12, and 21; (2) SEQ ID NOs 28, 29, and 34; and/or (3) SEQ IN NOs: 49, 55, and 56.

9. The method of claim 1, wherein measuring the plural of signature peptides comprises calculating corresponding peak heights or peak areas.

10. The method of claim 1, wherein measuring the plural of signature peptides comprises comparing data from high fragmentation mode and low fragmentation mode.

11. A high-throughput system for quantitating one or more protein of interest with known amino acid sequence in a plant-based sample, the system comprising:
(a) a high-throughput means for extracting proteins from a plant-based sample;
(b) a separation module for separating peptides in a single step;
(c) a selection module for selecting a plural of signature peptides from the protein of interest with known amino acid sequence; and
(d) a high resolution accurate mass spectrometry (HRAM MS) for measuring the plural of signature peptides.

12. The system of claim 11, wherein the separation module comprises a column chromatography.

13. The system of claim 11, wherein the high resolution accurate mass spectrometry (HRAM MS) does not comprise a tandem mass spectrometer.

14. The system of claim 11, wherein the plant-based sample is from a transgenic plant.

15. The system of claim 11, wherein the one or more protein of interest comprises 5-enolpyruvylshikimate-3-phosphate synthase (2mEPSPS), aryloxyalkanoate dioxygenase-12 (AAD-12), and/or phosphinothricin acetyltransferase (PAT).

16. The system of claim 17, wherein the plural of signature peptides comprises at least three sequences selected from the group consisting of SEQ ID NOs: 2-25, 27-45, and 47-60.

17. The system of claim 17, wherein the plural of signature peptides comprises (1) SEQ ID NOs 3, 12, and 21; (2) SEQ ID NOs 28, 29, and 34; and/or (3) SEQ IN NOs: 49, 55, and 56.

18. The system of claim 17, wherein the plural of signature peptides consist of (1) SEQ ID NOs 3, 12, and 21; (2) SEQ ID NOs 28, 29, and 34; and/or (3) SEQ IN NOs: 49, 55, and 56.

19. A high-throughput method of quantitating one or more protein of interest with known amino acid sequence in a plant-based sample, comprising using the system of claim 11.


SEQUENCE LISTING (60 sequences)

SEQ ID NO: 1 - protein, 445 aa, Arabidopsis thaliana
MAGAEEIVLQPIKEISGTVKLPGSKSLSNRILLLAALSEGTTVVDNLLNSEDVHYMLGALRTLGLSVEADKAAKRAVVVGCGGKFPVEDAKEEVQLFLGNAGIAMRSLTAAVTAAGGNATYVLDGVPRMRERPIGDLVVGLKQLGADVDCFLGTDCPPVRVNGIGGLPGGKVKLSGSISSQYLSALLMAAPLALGDVEIEIIDKLISIPYVEMTLRLMERFGVKAEHSDSWDRFYIKGGQKYKSPKNAYVEGDASSASYFLAGAAITGGTVTVEGCGTTSLQGDVKFAEVLEMMGAKVTWTETSVTVTGPPREPFGRKHLKAIDVNMNKMPDVAMTLAVVALFADGPTAIRDVASWRVKETERMVAIRTELTKLGASVEEGPDYCIITPPEKLNVTAIDTYDDHRMAMAFSLAACAEVPVTIRDPGCTRKTFPDYFDVLSTFVKN

SEQ ID NO: 2 - protein, 12 aa, Artificial Sequence
  Candidate signature peptide
AGAEEIVLQPIK

SEQ ID NO: 3 - protein, 7 aa, Artificial Sequence
  Candidate signature peptide
EISGTVK

SEQ ID NO: 4 - protein, 31 aa, Artificial Sequence
  Candidate signature peptide
ILLLAALSEGTTVVDNLLNSEDVHYMKGALR

SEQ ID NO: 5 - protein, 10 aa, Artificial Sequence
  Candidate signature peptide
TLGLSVEADK

SEQ ID NO: 6 - protein, 9 aa, Artificial Sequence
  Candidate signature peptide
AVVVGCGGK

SEQ ID NO: 7 - protein, 7 aa, Artificial Sequence
  Candidate signature peptide
FPVEDAK

SEQ ID NO: 8 - protein, 15 aa, Artificial Sequence
  Candidate signature peptide
EEVQLFLGNAGIAMR

SEQ ID NO: 9 - protein, 22 aa, Artificial Sequence
  Candidate signature peptide
SLTAAVTAAGGNATYVLDGVPR

SEQ ID NO: 10 - protein, 12 aa, Artificial Sequence
  Candidate signature peptide
ERPIGDLVVGLK

SEQ ID NO: 11 - protein, 18 aa, Artificial Sequence
  Candidate signature peptide
QLGADVDCFLGTDCPPVR

SEQ ID NO: 12 - protein, 11 aa, Artificial Sequence
  Candidate signature peptide
VNGIGGLPGGK

SEQ ID NO: 13 - protein, 31 aa, Artificial Sequence
  Candidate signature peptide
LSGSISSQYLSALLMAAPLALGDVEIEIIDK

SEQ ID NO: 14 - protein, 12 aa, Artificial Sequence
  Candidate signature peptide
LISIPYVEMTLR

SEQ ID NO: 15 - protein, 9 aa, Artificial Sequence
  Candidate signature peptide
AEHSDSWDR

SEQ ID NO: 16 - protein, 40 aa, Artificial Sequence
  Candidate signature peptide
NAYVEGDASSASYFLAGAAITGGTVTVEGCGTTSLQGDVK

SEQ ID NO: 17 - protein, 11 aa, Artificial Sequence
  Candidate signature peptide
FAEVLEMMGAK

SEQ ID NO: 18 - protein, 15 aa, Artificial Sequence
  Candidate signature peptide
VTWTETSVTVTGPPR

SEQ ID NO: 19 - protein, 8 aa, Artificial Sequence
  Candidate signature peptide
AIDVNMNK

SEQ ID NO: 20 - protein, 22 aa, Artificial Sequence
  Candidate signature peptide
MPDVAMTLAVVALFADGPTAIR

SEQ ID NO: 21 - protein, 6 aa, Artificial Sequence
  Candidate signature peptide
DVASWR

SEQ ID NO: 22 - protein, 19 aa, Artificial Sequence
  Candidate signature peptide
LGASVEEGPDYCIITPPEK

SEQ ID NO: 23 - protein, 13 aa, Artificial Sequence
  Candidate signature peptide
LNVTAIDTYDDHR

SEQ ID NO: 24 - protein, 18 aa, Artificial Sequence
  Candidate signature peptide
MAMAFSLAACAEVPVTIR

SEQ ID NO: 25 - protein, 14 aa, Artificial Sequence
  Candidate signature peptide
TFPDYFDVLSTFVK

SEQ ID NO: 26 - protein, 293 aa, Delftia acidovorans
MAQTTLQITPTGATLGATVTGVHLATLDDAGFAALHAAWLQHALLIFPGQHLSNDQQITFAKRFGAIERIGGGDIVAISNVKADGTVRQHSPAEWDDMMKVIVGNMAWHADSTYMPVMAQGAVFSAEVVPAVGGRTCFADMRAAYDALDEATRALVHQRSARHSLVYSQSKLGHVQQAGSAYIGYGMDTTATPLRPLVKVHPETGRPSLLIGRHAHAIPGMDAAESERFLEGLVDWACQAPRVHAHQWAAGDVVVWDNRCLLHRAEPWDFKLPRVMWHSRLAGRPETEGAALV

SEQ ID NO: 27 - protein, 63 aa, Artificial Sequence
  Candidate signature peptide
MAQTTLQITPTGATLLGATVTGVHLATLDDAGFAALHAAWLQHALLIFPGQHLSNDQQITFAK

SEQ ID NO: 28 - protein, 6 aa, Artificial Sequence
  Candidate signature peptide
FGAIER

SEQ ID NO: 29 - protein, 13 aa, Artificial Sequence
  Candidate signature peptide
IGGGDIVAISNVK

SEQ ID NO: 30 - protein, 6 aa, Artificial Sequence
  Candidate signature peptide
ADGTVR

SEQ ID NO: 31 - protein, 12 aa, Artificial Sequence
  Candidate signature peptide
QHSPAEWDDMMK

SEQ ID NO: 32 - protein, 35 aa, Artificial Sequence
  Candidate signature peptide
VIVGNMAWHADSTYMPVMAQGAVFSAEVVPAVGGR

SEQ ID NO: 33 - protein, 7 aa, Artificial Sequence
  Candidate signature peptide
TCFADMR

SEQ ID NO: 34 - protein, 11 aa, Artificial Sequence
  Candidate signature peptide
AAYDALDEATR

SEQ ID NO: 35 - protein, 6 aa, Artificial Sequence
  Candidate signature peptide
ALVHQR

SEQ ID NO: 36 - protein, 9 aa, Artificial Sequence
  Candidate signature peptide
HSLVYSQSK

SEQ ID NO: 37 - protein, 28 aa, Artificial Sequence
  Candidate signature peptide
LQHVQQAGSAYIGYGMDTTATPLRPLVK

SEQ ID NO: 38 - protein, 14 aa, Artificial Sequence
  Candidate signature peptide
VHPETGRPSLLIGR

SEQ ID NO: 39 - protein, 15 aa, Artificial Sequence
  Candidate signature peptide
HAHAIPGMDAAESER

SEQ ID NO: 40 - protein, 14 aa, Artificial Sequence
  Candidate signature peptide
FLEGLVDWACQAPR

SEQ ID NO: 41 - protein, 17 aa, Artificial Sequence
  Candidate signature peptide
VHAHQWAAGDVVVWDNR

SEQ ID NO: 42 - protein, 5 aa, Artificial Sequence
  Candidate signature peptide
CLLHR

SEQ ID NO: 43 - protein, 7 aa, Artificial Sequence
  Candidate signature peptide
AEPWDFK

SEQ ID NO: 44 - protein, 6 aa, Artificial Sequence
  Candidate signature peptide
VMWHSR

SEQ ID NO: 45 - protein, 13 aa, Artificial Sequence
  Candidate signature peptide
LAGRPETEGAALV

SEQ ID NO: 46 - protein, 183 aa, Streptomyces viridochromogenes
MSPERRPVEIRPATAADMAAVCDIVNHYIETSTVNFRTEPQTPQEWIDDLERLQDRYPWLVAEVEGVVAGIAYAGPWKARNAYDWTVESTVYVSHRHQRLGLGSTLYTHLLKSMEAQGFKSVVAVIGLPNDPSVRLHEALGYTARGTLRAAGYKHGGWHDVGFWQRDFELPAPPRPVRPVTQI

SEQ ID NO: 47 - protein, 5 aa, Artificial Sequence
  Candidate signature peptide
MSPER

SEQ ID NO: 48 - protein, 32 aa, Artificial Sequence
  Candidate signature peptide
RPVEIRPATAADMAAVCDIVNHYIETSTVNFR

SEQ ID NO: 49 - protein, 15 aa, Artificial Sequence
  Candidate signature peptide
TEPQTPQEWIDDLER

SEQ ID NO: 50 - protein, 4 aa, Artificial Sequence
  Candidate signature peptide
LQDR

SEQ ID NO: 51 - protein, 22 aa, Artificial Sequence
  Candidate signature peptide
YPWLVAEVEGVVAGIAYAGPWK

SEQ ID NO: 52 - protein, 16 aa, Artificial Sequence
  Candidate signature peptide
NAYDWTVESTVYVSHR

SEQ ID NO: 53 - protein, 13 aa, Artificial Sequence
  Candidate signature peptide
LGLGSTLYTHLLK

SEQ ID NO: 54 - protein, 8 aa, Artificial Sequence
  Candidate signature peptide
SMEAQGFK

SEQ ID NO: 55 - protein, 15 aa, Artificial Sequence
  Candidate signature peptide
SVVAVIGLPNDPSVR

SEQ ID NO: 56 - protein, 10 aa, Artificial Sequence
  Candidate signature peptide
LHEALGYTAR

SEQ ID NO: 57 - protein, 4 aa, Artificial Sequence
  Candidate signature peptide
GTLR

SEQ ID NO: 58 - protein, 5 aa, Artificial Sequence
  Candidate signature peptide
AAGYK

SEQ ID NO: 59 - protein, 12 aa, Artificial Sequence
  Candidate signature peptide
HGGWHDVGFWQR

SEQ ID NO: 60 - protein, 17 aa, Artificial Sequence
  Candidate signature peptide
DFELPAPPRPVRPVTQI